(19) United States
(12) Patent Application Publication
(10) Pub. No.: US 2003/0139736 A1
(43) Pub. Date: Jul. 24, 2003

(54) Ophthalmic surgical microscope

(76) Inventor: Ulrich Sander, Rebstein (CH)

Correspondence Address: George L. Snyder, Jr., Hodgson Russ LLP, Suite 2000, One M&T Plaza, Buffalo, NY, 14203-2391, US

(21) Appl. No.: 10/341,111

(22) Filed: Jan. 13, 2003

(30) Foreign Application Priority Data
Jan. 23, 2002 (DE) 102 02 509.6

(51) Int. Cl. A61B 18/18

(52) U.S. Cl. 606/4

(57) ABSTRACT

The invention concerns an ophthalmic surgical microscope (2) that comprises an integrated refractor (20) which determines the optical data of a patient's eye (1) “in situ” during the operation and reports them to the surgeon (29) as necessary.

### CROSS REFERENCE TO RELATED APPLICATIONS

This application claims priority of the German patent application 102 02 509.6 filed Jan. 23, 2002 which is incorporated by reference herein.

### FIELD OF THE INVENTION

The invention concerns an ophthalmic surgical microscope having an apparatus for in-situ determination of the optical data of a patient's eye during the operation.

### BACKGROUND OF THE INVENTION

If the lens of a patient's eye is clouded or severely deformed, at present it is usual to determine the optical data of the eye before the operation by means of a refractor, for example using the “Welch Allyn SURESIGHT® refractor of the Welch Allyn Co., NY 13153 (US), cf. brochure SM2275 Rev. B. On the basis of the results obtained, the dimension of the intraocular lens (IOL) to be implanted is determined. The IOL is then inserted into the eye during the operation. After the healing process, i.e. a few days later, another measurement is made using a refractor, and the residual vision deficiency is determined and corrected, if necessary, with eyeglasses.

The inventor has recognized that these known systems are disadvantageous in terms of the following aspects:

a) Because the optical data can be determined before the operation and then only several days after the operation, the surgeon has no ability to make corrections during the operation, i.e. in some circumstances a considerable uncorrected vision deficiency of the patient's eye remains.

b) The surgeon cannot, without interrupting the operation, determine the optical data of the patient's eye and thus initiate appropriate corrective actions to minimize the residual vision deficiency.

c) There is therefore no direct quality control during the operation.

### SUMMARY OF THE INVENTION

It is the object of the invention to create an apparatus which makes it possible to determine the optical data of a patient's eye during the operation with no need to interrupt the surgical procedure itself.

This object is achieved by way of the apparatus described below:

A refractor (autorefractive generator and analyzer), which allows determination of the optical data of the patient's eye “in situ” (i.e. during the operation), is integrated into a standard surgical microscope. This is preferably done in the following manner:

The light generated by a refractor is reflected, via an optical system and e.g. a beam splitter, into the main beam path of a microscope, and projected onto the patient's eye. The refractor light reflected from the retina of the patient's eye is reflected out of the beam path of the microscope, once again e.g. by means of a beam splitter, and imaged via an optical system onto an analysis unit of the refractor. The refractor and its analysis unit thus continuously determine the quality of the correction resulting from the lens implanted into the eye, and report it to the operator via a display, an apparatus for reflecting in data, and/or a signal tone or signal light. The refractor beam paths are reflected in and out, for example, directly above the zoom or between the zoom and the main objective. A number of variants can be constructed using the basic principle of the invention.

The following improvements are achieved by way of the apparatus described above:

Because the optical data of the patient's eye are determined during the operation, the surgeon can promptly initiate countermeasures if deviations from reference values occur.

Because potential deviations are measured and corrected promptly, the vision deficiency that remains after the healing process can be minimized.

All in all, the quality of the surgical result and the risk of a major deviation during the operation can therefore be reduced to a minimum.

According to a development of the invention, in the event of deviations exceeding a definable threshold the surgeon can be informed of an acute risk during the operation by means of a warning mechanism, for example a signal tone and/or a flashing light and/or a reflecting-in module.

### BRIEF DESCRIPTION OF THE DRAWINGS

The invention will be explained in more detail with reference to schematic drawings, in which:

FIG. 1 shows the integration, according to the present invention, of a refractor into a surgical microscope; and

FIG. 2 is a schematic block diagram of all the components.

### DETAILED DESCRIPTION OF THE INVENTION

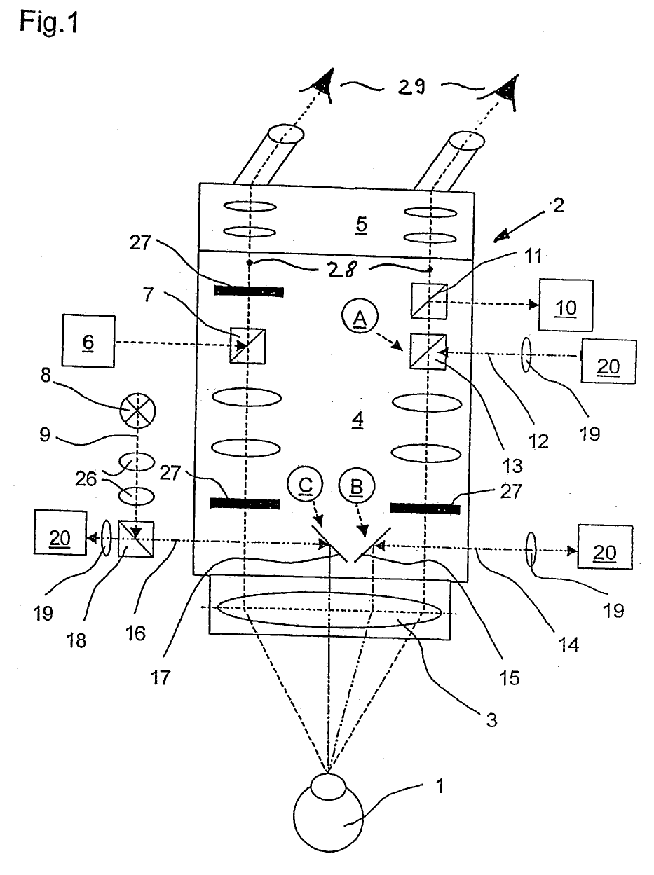

FIG. 1 shows the light emerging from patient's eye 1, which is directed via a main objective 3 and a zoom 4 onto tube 5 with eyepieces. Arranged in the right-hand main beam path of microscope 2, for example between zoom 4 and tube 5, is a beam splitter 11 which reflects out light for a video documentation apparatus 10. Arranged in the left-hand main beam path, for example between zoom 4 and tube 5, is a beam splitter 7 for reflecting data generated by an image superimposition apparatus 6 into the surgeon's field of view. Also visible is an illumination beam path 9, proceeding from an illumination system 8, which projects light via a beam splitter 18 and a deflection mirror 17 onto patient's eye 1.

According to the present invention, the light generated by a refractor 20 is superimposed via an optical system 19 for refractor beam path 12 at position A (located directly above zoom 4), by means of beam splitter 13, e.g. into the right-hand main beam path of microscope 2. This light is imaged, via zoom 4 and main objective 3, onto the retina of patient's eye 1. The refractor light reflected therefrom is in turn directed via main objective 3 and zoom 4 onto beam splitter 13, which reflects the refractor light out and directs it, via optical system 19 for refractor beam path 12, onto the measurement unit of refractor 20 for analysis.

As a variant, the refractor light is projected onto patient's eye 1 via a deflection mirror 15 for refractor beam path 14 at position B which lies directly above main objective 3, and recorded.

As a further variant, the refractor light is deflected at position C, which lies centrally above main objective 3 in alignment with a central optical axis of the objective lens, by means of deflection mirror 17 for refractor beam path 16 and illumination 9 with illuminating optical system 26, onto beam splitter 18 which combines illumination beam path 9 and refractor beam path 16. The refractor beam path is directed onto refractor 20 via an optical system 19 for the refractor beam path.

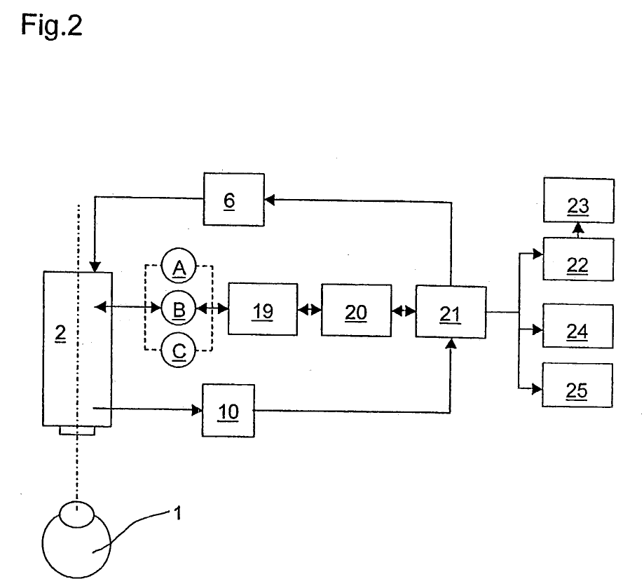

FIG. 2 is a schematic block diagram of all the components. The data measured by refractor 20 are processed by an analysis and control unit 21 and stored. Data output is accomplished, for example, via a monitor 22 on which patient's eye 1 is depicted, optionally with the optical data of the patient's eye measured by the refractor, and possibly with further patient data. The latter can in turn be printed out via a graphics printer 23 or a simple data printer 24, or can be transferred into patient file 25. The patient data required for the operation are transferred, as necessary, from patient file 25 into analysis and control unit 21.

Analysis and control unit 21 processes the data of video documentation apparatus 10, preferably a digital camera, and makes them available to the operator (29) via an image superimposition apparatus 6 for reflecting in data.

By means of shutters 27 (cf. FIG. 1) it is possible to darken, for example, the observation beam path (28) into which data are not being reflected. Optionally, however, this can also be done in the beam path into which the data are being reflected. This permits a better and higher-contrast data display. The capabilities disclosed in commonly owned U.S. patent application Ser. No. 10/081,154 published Sep. 12, 2002 under number US 2002/0126375 A1, which is incorporated herein by reference, are suitable for this purpose.

### PARTS LIST

1 Patient's eye

2 Surgical microscope

3 Main objective

4 Zoom

5 Tube with eyepieces

6 Image superimposition apparatus

7 Beam splitter for (6)

8 Illumination

9 Illumination beam path

10 Video documentation apparatus

11 Beam splitter for (10)

12 Refractor beam path at position A (reflection in and out)

13 Beam splitter for (12)

14 Refractor beam path at position B (reflection in and out)

15 Deflection mirror for (14)

16 Refractor beam path at position C (reflection in and out)

17 Deflection mirror for (16) and (8)

18 Beam splitter for (16)

19 Optical system for refractor beam path (positions A, B and/or C)

20 Refractor

21 Analysis and control unit

22 Monitor

23 Graphics printer

24 Data printer

25 Patient file

26 Illuminating optical system

27 Shutter (stop(s))

28 (Partial) beam paths for (29)

29 Observer (surgeon)

What is claimed is:

1. An ophthalmic surgical microscope for observing a patient's eye during surgery, the ophthalmic surgical microscope comprising:
a microscope optical path
a main objective lens in the microscope optical path;
an eyepiece tube in the microscope optical path;
a zoom system in the microscope optical path between the main objective lens and the eyepiece tube; and
refractor means for determining optical parameters of the patient's eye “in situ” during surgery and reporting the optical parameters to a surgeon, the refractor means having a refractor optical path coinciding in part with the microscope optical path.

2. The surgical microscope as defined in claim 1, wherein the refractor means generates a refractor beam traveling from the refractor means, and the surgical microscope includes an optical element for reflecting the refractor beam into the microscope optical path at at least one of the following three locations: (A) between the zoom system and the eyepiece tube, (B) between the objective lens and the zoom system, and (C) directly above the objective lens in alignment with a central optical axis of the objective lens.

3. The surgical microscope as defined in claim 1, wherein the refractor means generates a refractor beam that is reflected from the patient's eye for return to the refractor means, and the surgical microscope includes an optical element for reflecting the returning refractor beam out of the microscope optical path at at least one of the following three locations: (A) between the zoom system and the eyepiece tube, (B) between the objective lens and the zoom system, and (C) directly above the objective lens in alignment with a central optical axis of the objective lens.

4. The surgical microscope as defined in claim 2, wherein the optical element is chosen from a group consisting of a beam splitter, a prism, and a mirror.

5. The surgical microscope as defined in claim 3, wherein the optical element is chosen from a group consisting of a beam splitter, a prism, and a mirror.

6. The surgical microscope as defined in claim 4, wherein the optical element exclusively transmits and reflects light that is within a predetermined spectral wavelength region.

7. The surgical microscope as defined in claim 5, wherein the optical element exclusively transmits and reflects light that is within a predetermined spectral wavelength region.

8. The surgical microscope as defined in claim 1, further comprising a refractor optical system located in the refractor optical path adjacent the refractor means.

9. The surgical microscope as defined in claim 1, further comprising an optical element for reflecting light out of the microscope optical path, whereby the surgical microscope can be equipped with a video documentation apparatus which images the patient's eye.

10. The surgical microscope as defined in claim 1, further comprising an optical element for reflecting light into the microscope optical path, whereby the surgical microscope can be equipped with an image superimposition apparatus to provide surgical data in the surgeon's field of view.

11. The surgical microscope as defined in claim 1, wherein the surgical microscope includes an automatic focusing system and the refractor means is coupled bidirectionally to the automatic focusing system.

12. The surgical microscope as defined claim 1, further comprising an analysis and control unit having a memory means for storing the optical parameters of the patient's eye in a patient file.

13. The surgical microscope as defined in claim 12, further comprising a data printer connected to the analysis unit for printing the optical parameters of the patient's eye.

14. The surgical microscope as defined in claim 12, wherein the patient file includes data relevant for an operation, and the data are automatically accessed by the analysis and control unit.

15. The surgical microscope as defined in claim 10, further comprising a shutter in the microscope optical path between the optical element and the objective lens, the shutter being operable to selectively block light coming from the patient's eye for better visibility of the reflected-in surgical data.
